(19) United States
(12) Patent Application Publication
(10) Pub. No.: US 2013/0089932 A1
(43) Pub. Date: Apr. 11, 2013

(54) FET Sensor and Methods for Detecting Melamine

(71) Applicants: Qiang Wu, Plano, TX (US); Wenchuang Hu, Allen, TX (US); Ruhai Tian, Guilderland, NY (US)

(72) Inventors: Qiang Wu, Plano, TX (US); Wenchuang Hu, Allen, TX (US); Ruhai Tian, Guilderland, NY (US)

(21) Appl. No.: 13/648,396

(22) Filed: Oct. 10, 2012

Related U.S. Application Data
(60) Provisional application No. 61/545,239, filed on Oct. 10, 2011.

(51) Int. Cl. H01L 29/78; G01N 33/53

(52) U.S. Cl. 436/501; 257/253; 257/E29.255

(57) ABSTRACT

The present invention provides a device and methods for the detection and quantification melamine in a sample by rapid and specific electrochemical detection. The present invention includes using a field-effect transistor (FET) biosensor having an open Si channel with a melamine antigen, or hapten, or an antibody, anchored via a linker molecule such as self assembled monolayer to the surface of the gate dielectric of the said open Si channel. The anchoring molecule having the capability of detecting melamine directly or indirectly by selectively binding melamine antibodies, which changes a field-effect on a Si channel, causing a change in conductivity of the FET. This change in conductivity can be measured and is used to determine the presence or absence of melamine in a sample compared to a standard signal or pre-measured database.

### CROSS-RELATED TO RELATED APPLICATIONS

This application claims the benefit of U.S. Provisional Application No. 61/545,239, filed Oct. 10, 2011.

### FIELD OF THE INVENTION

The present invention relates generally to detection of melamine, and more particularly, to a sensor capable of immediate detection of melamine using a field-effect transistor (FET), and methods of detecting melamine using an FET.

### BACKGROUND OF THE INVENTION

Melamine (1,3,5-Triazine-2,4,6-triamine) has uses in several industrial areas, including the making of pesticides, fire retardants, concrete, and resins. While in low doses, melamine is non-toxic, in higher doses, melamine has been shown to be toxic in animals. Studies have shown that melamine causes skin irritation, renal failure, kidney stones, bladder stones, and reproductive damage.

While melamine can enter food sources by industrial leaching into the food supply, melamine has also been used as an adulterant in foodstuffs due to its nitrogen content, which yields false high protein readings when food is tested. This adulteration has led to several scandals involving melamine contamination. These scandals include the 2007 Chinese animal feed recall and the 2008 Chinese contaminated infant formula recall, where several children died from drinking milk contaminated with melamine. Melamine has been used as an adulterant because the nitrogen in melamine gives false high protein content readings, and companies wishing to increase the perceived protein content may add melamine to the foodstuff instead of actually increasing the protein content. Thus, the ability to detect the presence and amount of melamine has high importance in the food industry.

To date, there have been a number of methods to detect melamine, including the use of various mass spectrometry (MS) techniques, including High Performance Liquid Chromatography (HPLC), gas chromatography (GC), and Ultraviolet mass spectrometry (UVMS). Enzyme-linked immunosorbent assays (ELISA), and enzymatic detection methods are other methods currently used to detect melamine. However, mass spectrometry can be very expensive and time consuming. ELISA assays for melamine are time consuming, take several steps before melamine can be detected, and suffer from low accuracy of detection due to the low molecular weight of melamine. These techniques are often not conducive to quick accurate on-site detection of melamine, which is crucial, due to the short shelf life of milk products and other foodstuffs.

Common to almost all melamine tests is the use of a melamine antibody that binds to a melamine molecule. To produce a melamine antibody, melamine can be injected into a host animal. However, melamine is a small molecule and a weak melamine antigen which generates a weak or no immune response when injected into a host animal by itself. To overcome this lack of immune response, to generate high quality melamine antibodies, melamine is first attached to a hapten such as bovine serum albumin (BSA) to form a more powerful melamine antigen which then produces a melamine antibody. When the BSA-melamine protein is injected into a host animal, the immune system generates a vigorous response to the BSA-melamine antigen thereby generating high quality antibodies. Antibodies generated in this manner typically bind with high selectivity and specificity to the BSA-melamine antigen to form an antibody plus hapten-antigen complex. This antibody also binds with high selectivity and specificity to free melamine molecules to form the antibody plus antigen complex. Even though other similar molecules may be present in the sample, the melamine antibody binds selectively to only the melamine molecule. One type of melamine hapten based on BSA is Bovine Serum Albumin Sulfamethazine (BSA-SM2).

While there have been several methods and devices to detect small molecules, electronic sensors such as bio-FETs have shown great potential to achieve inexpensive and portable detection methods. An FET sensor works to detect biomolecules by using an electric field to control the charge carrier density on a semiconducting channel of the FET device. The key difference of an FET sensor from a typical FET device is that the top gate is removed so as to expose the semiconducting channel with gate dielectrics to the target sample, such as milk to be tested. Immobilized onto the surface of gate dielectrics (typically silicon dioxide) around the semiconducting channel of the FET sensor are probe molecules specific to target molecules. The target molecules bound to the channel of FET sensor can modulate the charge carrier density of the channel and therefore the change the conductance of the FETs via field-effects. A change in conductivity therefore indicates the presence of particular target molecules that bind to the probe molecules anchored on the surface of the semiconducting channel of the FET sensor.

One advantage of an FET sensor compared to other methods to detect biomolecules is that the small size of the semiconducting channel of the FET sensors provides higher detection sensitivity as it requires less target molecules to yield a measurable signal. For example, FET sensors with nanoscale channels have been proven to provide extremely high sensitivity in biochemical detection. An example of a nanoscale FET sensor in a device is a bio-fin-shaped Field Effect Transistor (bio-finFET) such as the one disclosed in PCT Application Publication No. WO 2012/050873 to Hu et al., incorporated herein by reference in its entirety.

Another type of nanoscale FET sensor devices is described as a nanogrid finFET in U.S. patent application Ser. No. 13/590,597 to Wu, incorporated herein by reference in its entirety. Nanoscale FET sensors such as finFET biosensors have been shown to be capable of measuring the concentration of proteins in solution down to the femto molar range. The fin channels of the finFET transistor have a high surface area which provides a high transistor channel area and high sensor sensitivity. A thin layer of SiO₂ as a gate dielectric is grown around the fins. An antibody to a target molecule may be attached to the gate dielectric covering the surface of the finFET transistor channel forming a sensor area. When the sensor area of the finFET transistor is immersed in a sample containing the target molecule, the target molecule binds to the antibody forming an antibody-target molecule complex. The change in charge caused by the formation of the antibody-target molecule complex changes the charge on a gate of the finFET transistor resulting in a change in conductance of the finFET transistor channel. The change in finFET transistor conductance may be measured by monitoring a transistor signal such as drive current (I_{ds}) and may be correlated to the amount of target molecule that is bound to the antibody on the gate. A sample with a low concentration of the target molecule will form few antibody-target molecule complexes resulting in a small change in the finFET transistor signal whereas a sample with a high concentration of the target molecule will form many antibody-target molecule complexes resulting in a large change in the finFET transistor signal.

Despite the available methods and devices to currently detect melamine, portable low cost sensors and methods to accurately detect low concentrations of are still desired.

### SUMMARY OF THE INVENTION

Embodiments of the present disclosure relate to devices and methods of melamine detection and/or quantification. Briefly described, embodiments of the present disclosure can include devices and methods of using a nanoscale silicon FET sensor, for detection of melamine based on a competitive antibody binding assay and a direct assay using antibody or aptamer.

In one embodiment of the invention, the sensor and the methods of detection of melamine involve a bio-FET such as a finFET biosensor. The FET biosensor comprises a semiconducting substrate and at least one open silicon channel on the semiconducting substrate. Attached to the gate dielectric on the silicon channel is a linker molecule (such as a silane based linker molecule) which is attached to the gate dielectric. The linker molecule (which may be a complex molecule formed via multiple steps of surface treatment) is also attached to a melamine antigen and forms the sensor area for measuring a presence of a target molecule that binds to the melamine antigen. In one embodiment, the melamine antigen is a melamine molecule, while in another embodiment the melamine antigen is a hapten melamine molecule such as BSA-SM2. The biosensor has circuitry arranged to measure a change in electrical signals passing through the FET. In one embodiment the biosensor is a finFET and in still another embodiment the biosensor is a nanogrid finFET.

When the target molecule binds to the melamine antigen on the gate dielectric, a change of the charge carrier density of the open silicon channel occurs, which changes the conductance of the FET biosensor via field effects. By changing the conductance of the FET, this allows the user of the device to determine the presence of melamine by measuring the change in the conductance of the FET.

In another embodiment of the present invention, instead of a melamine antigen bound to the gate dielectric (via the linker molecule) a melamine antibody is bound to the gate dielectric. The melamine antibody can bind melamine or a melamine hapten, and this sensor can be used to measure melamine concentration with a direct assay or competitive binding assay.

Embodiments for methods of detecting melamine are disclosed. In one embodiment, a competitive binding assay to detect melamine is used to determine the concentration of melamine. The concentration of melamine is determined by mixing a standard sample of known concentration of melamine antibody with a target sample having an undetermined concentration of melamine. This mixture is the testing sample which is immersed on the sensor area. An electrical transistor signal is measured through the FET biosensor to determine a melamine concentration by comparing the testing sample signal to a standard signal. In other embodiments, the sensor is immersed in a standard sample and a standard sample is determined instead of merely referencing a standard signal.

In one embodiment of a method to determine the concentration of melamine in a sample using a competitive binding assay, a melamine analog is anchored and immobilized to the gate dielectric of the sensor and used as a probe for melamine antibodies. The immobilized probe may be a melamine hapten such as BSA-SM2, which has a chemical group (such as sulfamethazine) that mimics melamine (both BSA-SM2 and melamine have an NH₂ group off of a benzene ring) and therefore can bind with added melamine antibodies in the solution. The immobilized probe molecule, when not bound to melamine antibodies produces a measurable current or voltage in the FET sensor as a baseline signal, and produces a different measurable signal when bound to melamine antibodies forming a complex. This occurs due to charge differences between the bound and unbound immobilized molecule on the gate dielectric. The different field-effects created by either bound or unbound probe molecules on the gate dielectric produce different electrical signals when current passes through the FET.

A change of the number of melamine antibodies capable of binding to the immobilized molecule is directly proportional to the number of melamine molecules in a solution of melamine and melamine antibodies. By detecting a change in the electrical signals of the finFET sensor, the presence of melamine antibodies is determined. Due to competitive binding, when the presence of melamine antibodies is determined, the presence and concentration of melamine is also determined.

In another embodiment of detecting the presence of melamine, the binding assay has a step of mixing a first sample with a melamine antibody solution. The first sample has no presence of melamine and is used as a reference sample to obtain a baseline measurement. A second sample (having an undetermined amount of melamine), is mixed with the same melamine antibody solution as the test sample. The reference sample solution is applied to the sensing surface of the device and a baseline measurement of an electrical signal across the FET is obtained. The sensor is rinsed of the reference solution, and the second sample is applied to the sensing surface. A final measurement of an electrical signal of the FET is obtained for this sample to determine the presence of melamine. The presence of melamine in the test solution is determined by comparing the electrical signal obtained from the reference solution and the electrical signal after the test solution is applied. If there is no change in the electrical signal there is no presence of melamine in the test solution. If there is a difference between the two electrical signals (such as conductance), melamine is present in the test solution. A quantitative measurement of the concentration of melamine can be obtained by comparing the change of the FET signal to a pre-measured standard curve of signal levels vs. amount of melamine.

In another object of the invention, a similar competitive assay can be achieved by exchanging the roles of the probe molecule and the melamine antibody from the previously sensor embodiment. In other words, the melamine antibody can be anchored on the FET surface, while a known concentration of BSA-SM2 can be added to both the reference and the test solutions. Then melamine and BSA-SM2 will compete for binding with the antibodies on the FET surfaces. Since melamine is not charged, higher melamine concentration in the test solution can reduce binding of BSA-SM2 with the antibodies causing a larger change of FET signals from baseline. Lower melamine concentration yields smaller change from the baseline signals.

In yet another object of the invention, a direct detection assay can be achieved by using the FET sensor coupled with melamine antibodies to directly detect melamine. The binding of melamine to the antibody on the FET changes the charge of the antibody itself, causing a change of channel conductance of the FET sensor. Compared to competitive assay method, the direct assay is simpler but less sensitive.

Accordingly, an object of the invention is to provide a field-effect transistor device capable of detecting the presence of melamine and another object is to provide methods to detect the presence of melamine.

### BRIEF DESCRIPTION OF THE DRAWINGS

These and other features and advantages of the present invention will become appreciated as the same becomes better understood with reference to the specification, claims and drawings wherein:

FIG. 1A is a perspective view of a nanoscale FET sensor in the prior art.

FIG. 1B is a cross sectional view of antibodies binding to a target molecule on a gate dielectric covering the surface of a finFET.

FIG. 2 is an illustration of melamine antibodies, antigens, and non-melamine structures.

FIG. 3A is an illustration of a melamine antibody forming a hapten-antigen complex.

FIG. is an illustration of a melamine antibody forming the antibody plus antigen complex.

FIG. 4A is a cross sectional view of an FET sensor in a direct binding assay with a low concentration of melamine.

FIG. 4B is a cross sectional view of an FET sensor in a direct binding assay with a high concentration of melamine.

FIG. 5A is a cross sectional view of an FET sensor during a competitive binding assay according to an antigen anchored embodiment of the invention with a low concentration of melamine.

FIG. 5B is a cross sectional view of an FET sensor during a competitive binding assay according to an antigen anchored embodiment of the invention with a high concentration of melamine.

FIG. 6A is a cross sectional view of an FET sensor during a competitive binding assay according to a hapten anchored embodiment of the invention with a low concentration of melamine.

FIG. 6B is a cross sectional view of an FET sensor during a competitive binding assay according to a hapten anchored embodiment of the invention with a high concentration of melamine.

FIG. 7 is a graph of a melamine assay standard curve for determining melamine concentration using a finFET.

FIG. 8A is a graph showing experimental results of a BSA-SM2 treated finFET showing monotonic dependence of sensor signals vs. antibody concentration.

FIG. 8B is a graph showing competitive assay results of the finFET sensor of FIG. 8A.

FIG. 9A is a graph showing the experimental results of direct melamine detection using an antibody anchored finFET sensor.

FIG. 9B is a graph of a standard curve obtained for a direct assay of melamine.

### DETAILED DESCRIPTION OF THE EMBODIMENTS

It is to be understood that this disclosure is not limited to the particular embodiments described. It is also to be understood that the terminology used is for the purpose of describing particular embodiments only, and is not intended to be limiting, since the scope of the present disclosure will be limited only by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

The following examples are put forth so as to provide those of ordinary skill in the art with a complete disclosure and description of the device and how to perform the methods of detecting melamine. Unless otherwise stated, parts are by weight, temperatures in degrees Celsius (C.), and pressure is at or near atmospheric pressure. Standard temperature and pressure are defined as 20° C. and 1 atmosphere.

It must be noted that, as used in the specification and the appended claims, the singular forms “a,” “an,” and “the” include the plural references unless the context clearly dictates otherwise.

Unless defined otherwise, all terms used herein have the same meaning as commonly understood by one of ordinary skill in the art of which this disclosure belongs. Although any methods and material similar or equivalent to those described herein can also be used in the practice or testing of the present disclosure, the preferred methods and materials are now described.

The term “sample channel” refers to the area into which a sample is placed to come into contact with the sensor area of the finFET biosensor transistor. The sample channel may be a pipe like structure thorough which a sample flows or may be a sample well that may be filled with sample solution. A sample solution to be tested for melamine may flow through a conduit like sample channel and over the sensor area or a finFET biosensor device containing an opening such as a sample well may be immersed in a sample to be tested for melamine.

The term “finFET signal” refers to both the directly measured finFET transistor electrical parameters and also refers to parameters which may be derived from the measured finFET transistor electrical parameters. The detected signals of the transistor biosensor can be in many forms. For directly measured finFET signals, there can be several different biasing and configurations. One method is to bias the source and drain with a known voltage and also bias a gate electrode with another known voltage, measure the drain current during sensing experiments. Another method is to bias the source and drain with a current source and bias a gate electrode with a known voltage, and measure the drain voltage during sensing. A third method is to bias the source and drain with a known voltage, sweep the voltage of gate electrode in a chosen voltage range, to simultaneously measure the drain current, and to generate a standard transistor current versus voltage (I-V) plot.

The term “standard sample” or “standard solution” refers to a sample with a known concentration of melamine. The known concentration may be zero mg/ml or may be a nonzero mg/ml.

The term “reference sample” or “reference solution” refers to a sample solution with a known concentration of melamine antibody for the embodiment of competitive assays with melamine-hapten anchored sensor; or with a known concentration of melamine-hapten such as BSA-SM2 for the embodiment of competitive assays with melamine-antibody anchored sensor. The reference sample solution may have no melamine.

The term “target sample” refers to a sample with an unknown concentration of melamine.

The term “target signal” refers to a finFET signal measured either when the sensor area of a finFET biosensor transistor is immersed in a target sample or after the sensor area of a finFET biosensor transistor was immersed in a target sample.

The term “standard signal” refers to a finFET signal measured either when the sensor area of a finFET biosensor transistor is immersed in a standard sample or after the sensor area of a finFET biosensor transistor was immersed in a standard sample.

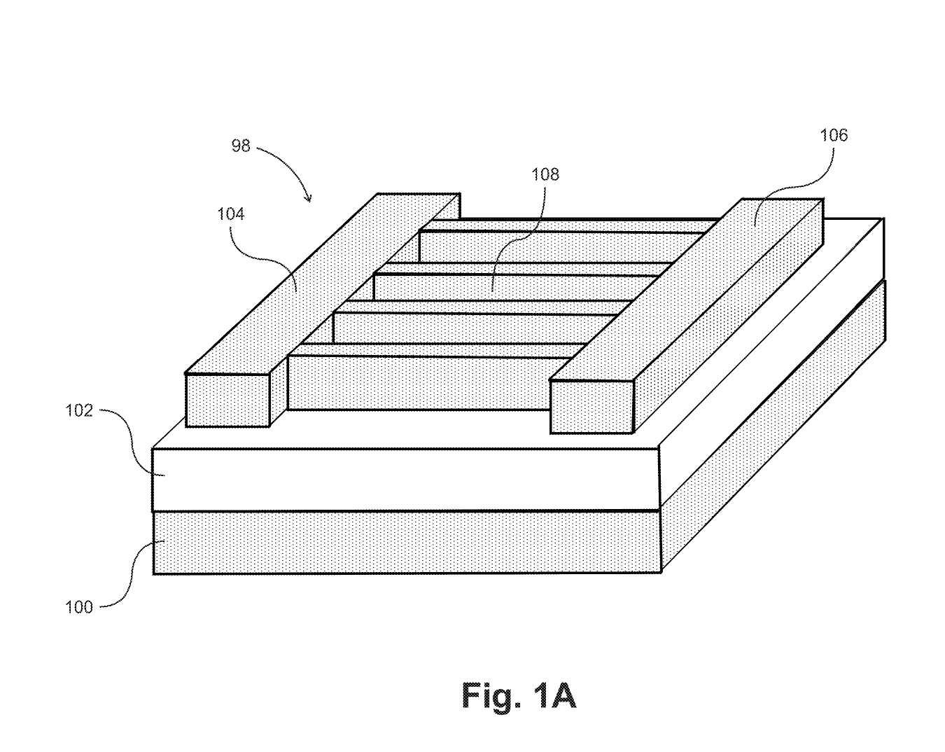

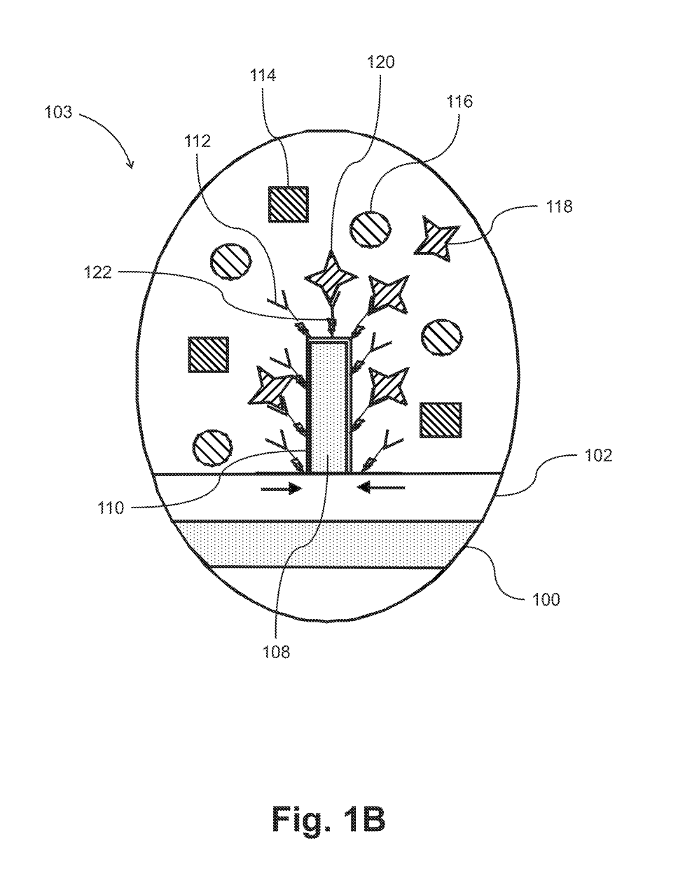

A finFET biosensor according to an embodiment is illustrated in FIG. 1A. The finFET biosensor transistor 98 consists of a source electrode 106, a drain electrode 104, with multiple silicon channel fins 108 forming parallel transistor channels between the source 106 and drain 104. The finFET biosensor is formed on a semiconductor on insulator (SOI) which consists of a substrate 100 which may be silicon, with a buried oxide (BOX) 102 electrically isolating the finFET biosensor from the substrate 100. A thin layer of SiO₂ or nitride SiO₂ as gate dielectrics 110 is grown around the fins 108. The probe molecule 112 is attached to the gate dielectrics 110 via a linker molecule 122. Silane based self assembled monolayers (SAMs) are often used as the linker molecule. Many times, multiple surface treatment processes may be needed to make the linker molecule or a complex to have desired functions for attaching probes. For simplicity, in the following figures and descriptions, the linker molecules 122 are not shown or described in subsequent illustrations and descriptions.

Contacts are formed to the source 106 and drain 104 of the finFET biosensor to measure an electrical property or signal of the finFET biosensor transistor such as drive current (I_{ds}). A sample solution flows over the channel area of the finFET biosensor. Surface areas in the sample channel outside the sensor area may be coated with anti-adhesion protective molecules such as polyelthylene glocol (PEG) terminated self assembled monolayers (SAMs), benzene terminated SAMs, fluorocarbon silanes, etc., which prevent melamine in the sample from adsorbing to non-sensor areas causing a change in the melamine concentration.

As shown in FIG. 1B, a thin layer of SiO₂ as gate dielectrics 110 is grown around the fins 108. Then an antibody 112 to a target molecule 118 may be attached to the gate dielectric 110 covering the surface of the finFET transistor channel 108 forming a sensor area. When the sensor area of the finFET transistor is immersed in a sample containing the target molecule 118, the target molecule binds to the antibody forming an antibody-target molecule complex 120. The change in charge caused by the formation of the antibody-target molecule complex 120 changes the charge on a gate of the finFET transistor resulting in a change in conductance of the finFET transistor channel. The change in finFET transistor conductance may be measured by monitoring a transistor signal such as drive current (I_{ds}) and may be correlated to the amount of target molecule that is bound to the antibody on the gate. A sample with a low concentration of the target molecule will form few antibody-target molecule complexes resulting in a small change in the finFET transistor signal whereas a sample with a high concentration of the target molecule will form many antibody-target molecule complexes resulting in a large change in the finFET transistor signal.

The finFET signal may also be indirect measurements or parameters derived from directly measured finFET transistor electrical parameters as outlined above. For example, the change in one of the measured finFET transistor electrical parameters may be derived by subtracting the initial measured finFET transistor electrical parameter measured before the sample is introduced into the sensor area from the finFET transistor electrical parameter measured after the sample is introduced into the sample area. A percentage change may additionally be derived by dividing the relative change by the initial value. Alternatively, the transistor conductance may be derived by dividing the measured finFET transistor drain current by the finFET transistor drain voltage, or the trans-conductance of the finFET transistor may be derived by dividing the measured finFET transistor drain current by the voltage of gate electrode. With the measured I-V curve of the finFET transistor, the finFET transistor threshold voltage (Vt) or change in Vt or shift in Vt, etc may also be extracted. These direct or indirect finFET biosensor transistor signals are examples of biosensor signals that may be used to analyze results and may be correlated to the concentration of melamine in the sample. Conductance of the transistor as an exemplary signal of the sensor device in the following embodiments, but other measurements of the transistor signals may be used to determine concentration of melamine.

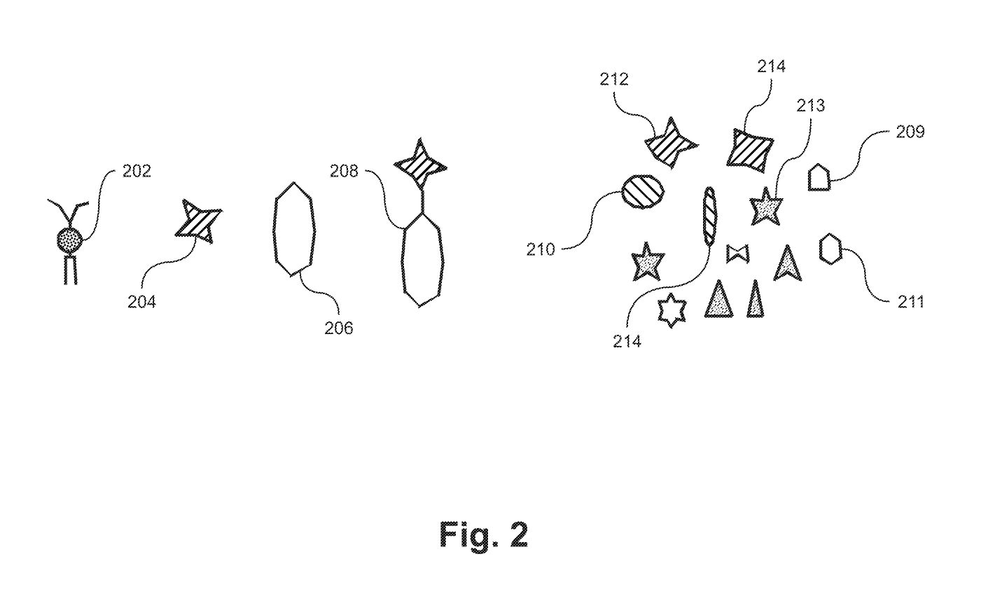

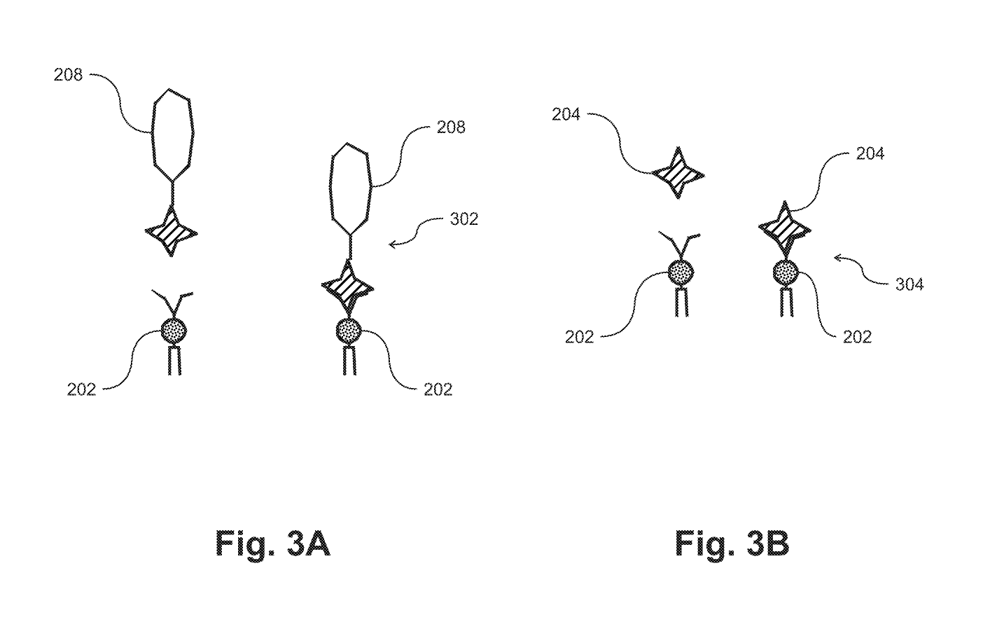

FIG. 2 and FIG. 3 illustrate representations of biomolecules in the detection of melamine. Typically, to produce a high quality melamine antibody, melamine 204 is first attached to a hapten 206 such as bovine serum albumin (BSA) to form a more powerful melamine antigen. When the BSA-melamine protein 208 is injected into a host animal, the immune system generates a vigorous response to the BSA-melamine antigen 208 generating high quality antibodies. Antibodies 202 generated in this manner typically bind with high selectivity and specificity to the BSA-melamine antigen 208 to form the antibody plus hapten-antigen complex 302 and also binds with high selectivity and specificity to free melamine molecules 204 to form the antibody plus antigen complex 304. Even though other similar molecules 209, 210, 211, 213, 214 may be present in the sample, the melamine antibody binds selectively to only the melamine molecule.

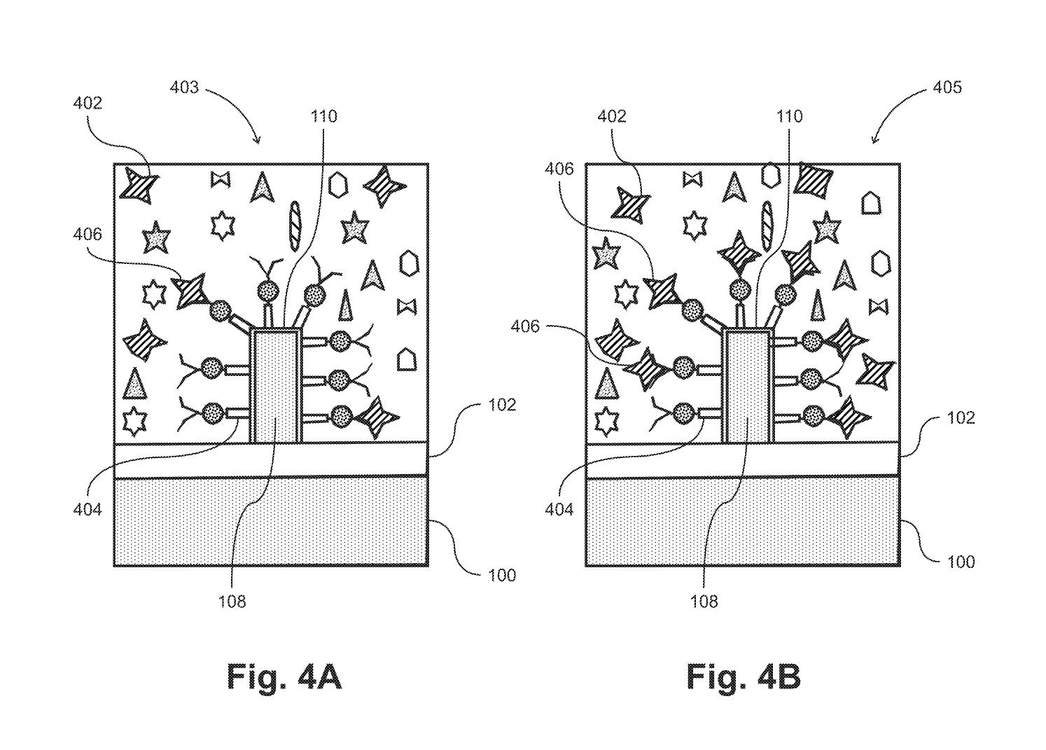

In one embodiment of bio-finFET sensors, FIG. 4A and FIG. 4B shows sensor areas 403, 405 and for detecting melamine using a direct detection assay. Antibodies 404 to melamine 402 are anchored to the gate dielectric 110 on the finFET sensor areas 403, 405. When a sample containing melamine comes into contact with the sensor area, the melamine antibody 404 immobilized on the finFET transistor channel 108 binds to the melamine molecule 402 forming a melamine antibody-melamine complex 406 that causes a change in channel conductance. If the sample contains a high concentration of melamine 404 as shown in FIG. 4B, more melamine antibody-melamine complexes 406 form on the finFET biosensor channel 108 causing a larger change in channel conductance. While this direct binding embodiment may be sufficient to detect whether melamine is present or absent in a sample, because melamine is a small uncharged molecule, the change in fin channel conductance when a melamine molecule 406 is bound by the immobilized melamine antibody 404 may be small and therefore the detection has a poor sensitivity in comparison to the competitive assay method. However, the direct detection method is a simpler method.

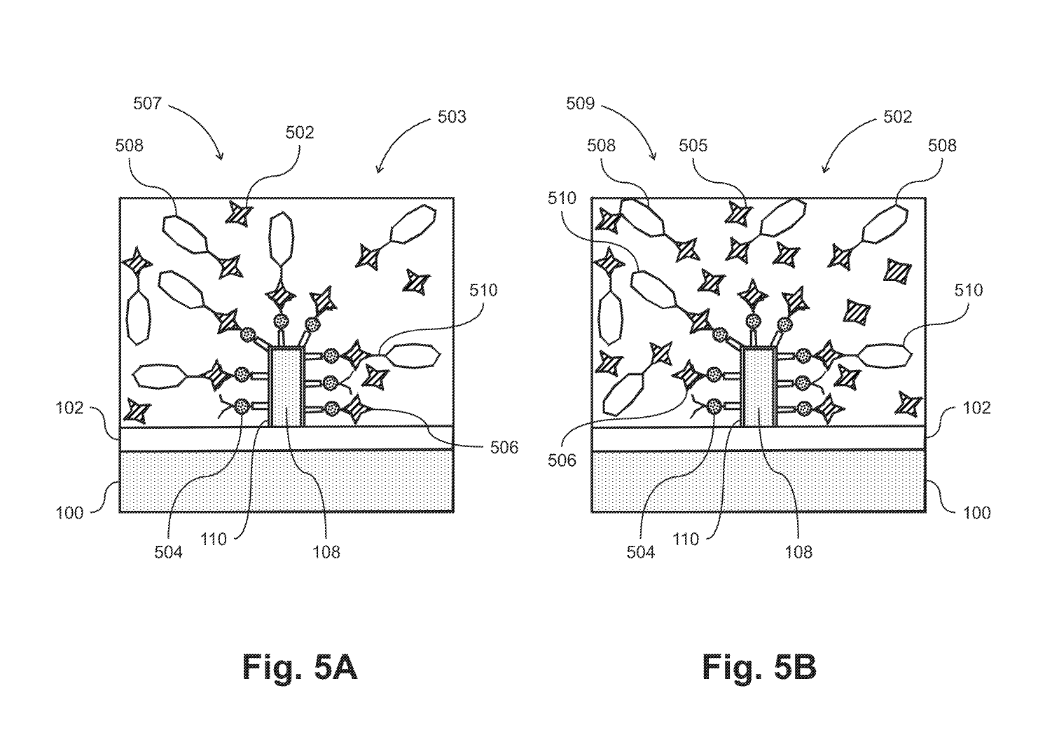

In an embodiment of sensors used for a competitive binding assay, as shown in FIG. 5A and FIG. 5B, a known concentration of hapten-melamine molecules such as BSA-SM2 508 may be added to the sample prior to immersing the finFET biosensor sensor area containing immobilized melamine antibody 504 in a sample solution. FIG. 5A illustrates a sensor area 507 with antibodies anchored to the gate dielectric 110 having a low concentration of melamine, while FIG. 5B illustrates a sensor area 509 with melamine antibodies 504 anchored to the gate dielectric 110 having a high concentration of melamine. In this embodiment, the hapten-melamine molecules 508 compete with the melamine molecules 502 in the sample solution for binding sites on the immobilized melamine antibody 504. If there is a low concentration of melamine molecules 502 in the sample solution, then most of the melamine antibody sites 504 will be bound to hapten-melamine molecules 510 as shown in FIG. 5A.

If, however, there is a high concentration of melamine molecules 502 in the sample solution, as in FIG. 5B, then most of the melamine antibody binding sites 504 will be bound to melamine molecules 506. Since the hapten-melamine molecule 508 carries significant charge, differences in the number of bound hapten-melamine molecules causes a larger change in finFET channel conductance than differences in the number of bound melamine molecules. Competitive binding of the hapten-melamine molecule thus increases the sensitivity of the finFET biosensor melamine assay.

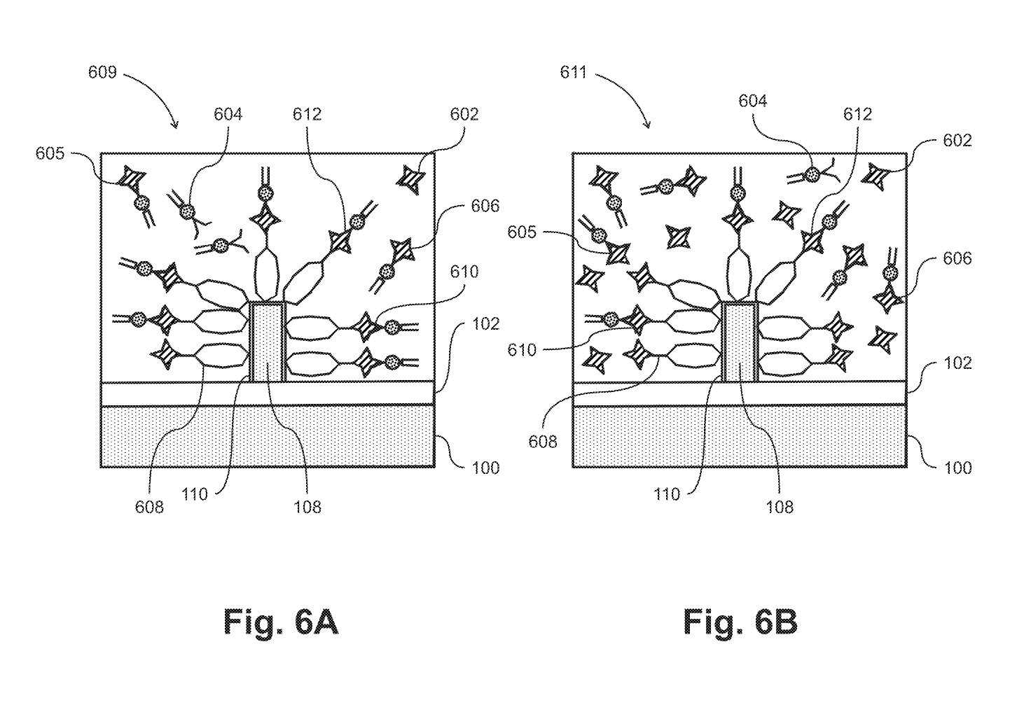

In another embodiment of a sensor and method depicted in FIGS. 6A and 6B, a competitive binding assay provides increased sensitivity for the detection of melamine by binding a molecule with a significant amount of charge to the finFET biosensor transistor channel 108. FIG. 6A illustrates a sensor area 609 during conditions of a low concentration of melamine and FIG. 6B illustrates a sensor area 611 during conditions of a high concentration of melamine. In this competitive binding embodiment the hapten-melamine molecule BSA-SM2 608 is be anchored to the finFET channel 108. A known concentration of melamine antibody 604 is added to a sample of unknown concentration of melamine to create a testing solution. The melamine antibodies 604 in the testing solution will competitively bind to the melamine molecules 602 in solution to form complex 606, and to the hapten-melamine molecules 608 immobilized on the finFET transistor channel 108 to form complex 612. If there is a low concentration of melamine 602 in the sample as shown in FIG. 6A a large number of the melamine antibody molecules 604 will bind to the hapten-melamine molecules immobilized on the finFET transistor channel 108 producing a particular electrically measured signal.

If, however, there is a high concentration of melamine 602 in the sample as shown in FIG. 6B, most of the melamine antibody molecules 604 will bind to melamine molecules 602 to form complex 606 in solution and few of the melamine antibody molecules 604 will be available to bind to the immobilized hapten-melamine molecules 608 to form a BSA-SM2 antibody complex 610. Since the melamine antibody 604 has significantly more charge than the melamine molecule, a change in amount of the melamine antibody 610 bound to the finFET transistor channel 108 causes a significantly larger change in channel conductance than does a change in the amount of melamine molecules bound to the finFET transistor channel 108. The number of BSA-SM2 antibody complexes 610 is inverse proportional to the amount of melamine in the testing solution, and can be measured via changes in field effects that occur when BSA-SM2 forms the complex 608 with melamine antibodies 604. Competitive binding of the melamine molecule thus increases the sensitivity of the finFET biosensor melamine assay

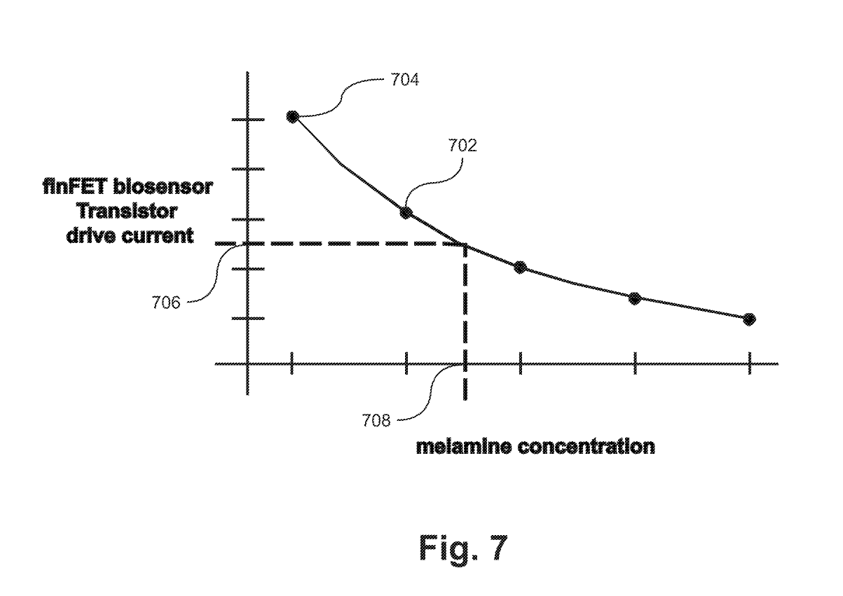

As shown in FIG. 7, a series of standard solutions with standard concentrations of melamine 702 may be used to generate a standard curve 704 of melamine concentration vs finFET transistor drive current. The concentration of melamine in an unknown sample 708 may then be determined by reading the drive current 706 from a sample off the standard curve 704.

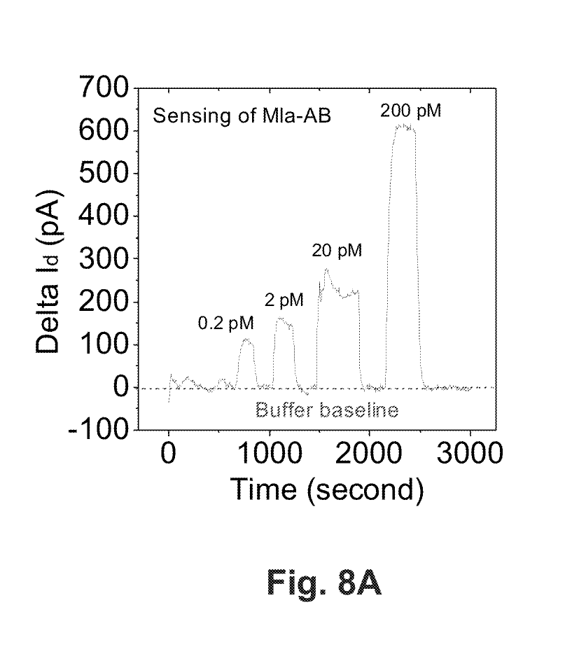

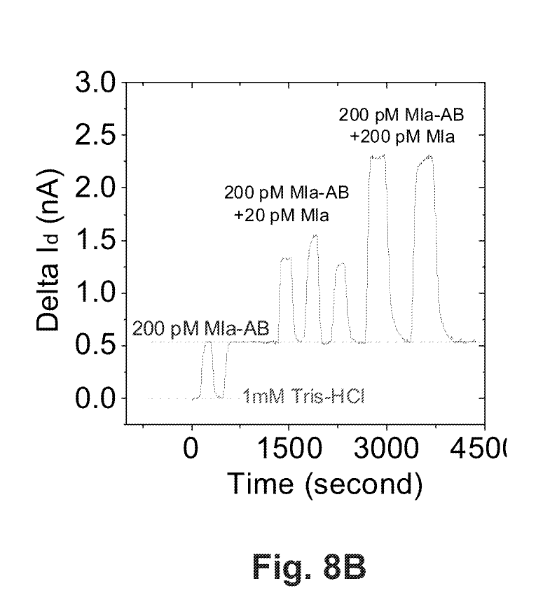

FIG. 8A shows the experimental results of BSA-SM2 treated finFET to different concentrations of melamine antibodies from 0.2 pM to 200 pM showing monotonic dependence of sensor signals vs. antibody concentration. This result demonstrates good binding between BSA-SM2 and melamine antibody. FIG. 8B shows the competitive assay results using the same finFET sensor of FIG. 8A. 200 pM of melamine antibodies is added to two target sample solutions (one with 20 pM melamine and one with 200 pM melamine). Test solution of 20 pM melamine (MLa) yields a small signal change from baseline while 200 pM melamine yields higher signal changes, demonstrating successful detection of melamine at a low detection limit (high sensitivity) using the competitive assay method. The assay sensitivity (limit of detection or LOD) achieved using this competitive assay is several orders of magnitude higher (lower for LOD) than conventional methods such as ELISA or mass spectrometry.

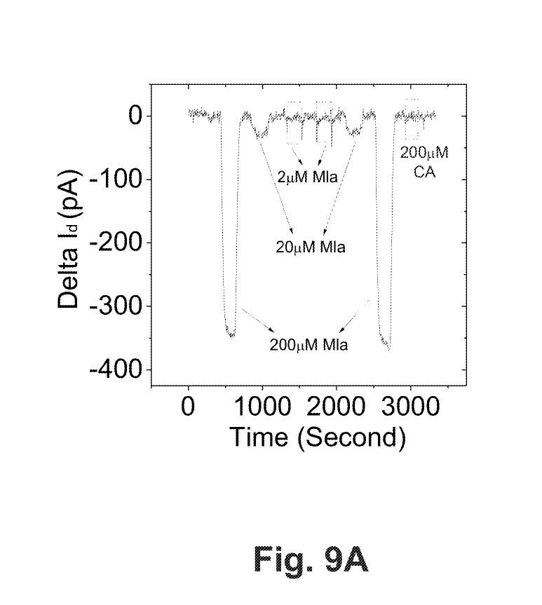

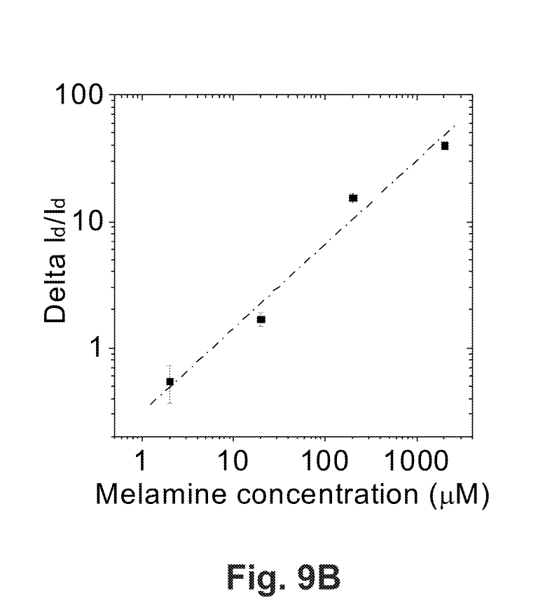

FIG. 9A shows the experimental results of direct detection of melamine using antibody anchored finFET sensor devices. A change of finFET current is found monotonic to the concentration of melamine, with higher melamine concentrations yield larger signal changes. It is noted that the solution of melamine concentration of 2 uM gives a very small signal, in comparison to FIG. 8B, showing the competitive assay provides much higher detection sensitivity than the direct detection method. FIG. 9B shows a standard curve obtained for direct assay experiments for the detection of melamine.

A method of detecting a presence of melamine in a sample using the modified sensors is now described. A sample known to have no presence of melamine is mixed and diluted into a 1 mM TRIS-HCL buffer pH 7.5 to produce a reference sample solution having no melamine. The solution is filtered through a 0.2 μm filter. Milk, or other foodstuff with possible but undetermined amount of melamine is mixed and diluted with the same amount of 1 mM TRIS-HCl buffer pH 7.5 solution that the baseline reference solution was mixed and diluted with to produce a test solution. For a competitive assay method, a known concentration of antibody (e.g. 200 pM) is added to both the reference and test solutions. As shown in FIG. 8B, first, the reference sample solution with 200 pM antibody is applied to the sensing surface of the FET with BSA-SM2 attached to the fin surfaces, and a first electrical signal is measured as a baseline. Then, the test solution is applied to the sensing surface and a second electrical signal is measured on the FET device. The presence of melamine in the test solution is determined by comparing the baseline reference measurement and the testing solution measurement. The difference in the first and second electrical signals in the presence of melamine is due to the competitive binding of melamine and the immobilized molecule (BSA-SM2) to melamine antibodies. When melamine binds to the melamine antibodies, melamine prevents the melamine antibodies from binding to the immobilized molecule (BSA-SM2). Since the immobilized molecule produces a different field-effect on the silicon nanochannel compared to when the immobilized molecule is bound to melamine antibodies, the conductivity and the electrical signals, such as drain current, as measured by the FET device, changes. As shown in FIG. 8B, 200 pM melamine causes higher change of current from the baseline than the 20 pM melamine to approve the feasibility of this method.

Because of the reproducibility of the finFET biosensor technology, a signal may be measured from a standard sample and the value of that signal may be stored in a data base and used as the reference value. For example, a target signal from a target sample containing an unknown amount of melamine may be compared with a standard signal from a database to determine the concentration of the melamine in the target sample without actually generating a standard signal by measuring a standard sample in the field.

### Sensor Preparation

The preparation of the sensor on the device to detect melamine is illustrated in the proceeding examples. Materials used in the preparation of the sensor are as follows:

| Chemical | Vendor | CAS/Cat |
|---|---|---|
| 3-aminopropyltriethoxysilane | Sigma-Aldrich | 919-30-2 |
| (APTES, >98%) | | |
| Triethoxysilyl undecanal | Gelest | 116047-42-8 |
| (TESU, >90%) | | |
| 11- | Gelest | 116821-45-5 |
| Aminoundecyltriethoxysilane | | |
| (AUTE >95%) | | |
| Anhydrous toluene (>99.8%) | Sigma-Aldrich | 108-88-3 |
| Anhydrous ethanol (>99.8%) | Sigma-Aldrich | 64-17-5 |
| Triethylamine (>99.8%) | Sigma-Aldrich | 121-44-8 |
| PEG-silane (MW = 2000) | Nanocs | PEG6-0102 |
| Sodium cyanoborohydride | Sigma-Aldrich | 25895-60-7 |
| Ethanol amine | Sigma-Aldrich | 141-43-5 |

Below, an example of a proven surface chemistry to prepare the finFET sensor for melamine detection is described in detail. FIG. 6 shows the functionalized sensor device for a competitive assay to detect melamine. The sensor comprises a silicon finFET with a fin surface modified to detect melamine in a sample. The surface of a gate dielectric (typically SiO₂) of Si fins is modified with silane molecules as linker molecule such as (3-aminopropyltriethoxysilate) (APTES) or Triethoxysilyl undecanal (TESU) to activate the fin surface for antibody immobilization. The silane molecules are attached to the sensing areas of the devices including the fins and the surrounding area of SiO₂. The channel or fin area is first cleaned with fresh piranha solution, a mixture of sulfuric acid (H₂SO₄) and hydrogen peroxide (H₂O₂) with a ratio of 1:1 for example, for 30 seconds or longer. The piranha cleaned chip can be stored in deionized (DI) water to maintain the surface cleanness and surface hydrophilicity for more than one month without any dissolution of oxide. An anhydrous solution with 0.1% TESU is mixed and ultrasonicated for 1 minute. The chip having the sensor is immersed in 0.1% anhydrous toluene solution for 1.5 hours. The sensor is rinsed with an excess of anhydrous solution. Melamine hapten BSA-SM2 is immobilized onto the fin surface for the first competitive assay (FIG. 6) by immersing the TESU functionalized fin surface in 50 mg/ml BSA-SM2 buffer solution (1 mM NaCNBH₃ in 2 mM potassium phosphate buffer pH 7.4) for 3 hours.

The same process can be used to anchor melamine antibody to the finFETs for another embodiment of a competitive assays (see description of FIG. 5). The modified silicon nanochannel or fins is rinsed in 2 mM potassium phosphate buffer pH 7.4 solution for 5 minutes to remove physically adsorbed antibody. The silicon finFETs is immersed in a 50 mM ethanolamine buffer solution (100 mM NaCNBH₃ in 2 mM potassium Phosphate buffer pH 7.4:5 mM ethanolamine at a 1:100 ratio) for 3 hours to passivate the unreacted aldehyde groups. The modified silicon fins are rinsed in 2 mM potassium phosphate buffer pH 7.4 for 5 minutes to remove physically adsorbed molecules.

While various embodiments have been described above, they are presented by way of example only and are not to be construed as a limitation of the invention. Numerous changes to the disclosed embodiments can be made without departing from the scope of the invention. The scope of the invention is defined in accordance with the following claims and their equivalents.

What is claimed is:

1. An FET biosensor comprising,
a semiconductor substrate;
at least one open Silicon channel integral with said semiconductor substrate;
a linker molecule;
a gate dielectric layer on a surface of said at least one open Silicon channel, said gate dielectric having a surface for attachment of said linker molecule; and,
a melamine antigen anchored to said gate dielectric via said linker molecule forming an FET sensor area for measuring a presence of a target molecule that binds to said melamine antigen;
whereby binding of said target molecule to said melamine antigen modulates the charge carrier density of said open Silicon channel, changing the conductance of said FET biosensor via field effects, thereby allowing a practitioner to determine a presence of melamine by measuring a change in conductance of said FET biosensor.

2. The FET biosensor of claim 1 wherein said FET is a finFET.

3. The FET biosensor of claim 1 wherein said FET is a nanogrid finFET.

4. The FET biosensor of claim 1 wherein said melamine antigen is a melamine molecule.

5. The FET biosensor of claim 1 wherein said melamine antigen is a hapten-melamine molecule.

6. The FET biosensor of claim 1, wherein said melamine antigen is a BSA-SM2 molecule.

7. An FET biosensor comprising,
a semiconductor substrate;
at least one open Silicon channel integral with said semiconductor substrate;
a linker molecule;
a gate dielectric layer on a surface of said at least one open Silicon channel, said gate dielectric having a surface for attachment of said linker molecule; and,
a melamine antibody anchored to said gate dielectric via said linker molecule, forming an FET sensor area.

8. The FET biosensor of claim 7 wherein said FET is a finFET.

9. The FET biosensor of claim 7 wherein said FET is a nanogrid finFET.

10. The FET biosensor of claim 7 wherein said melamine antibody comprises a binding region capable of binding a melamine molecule.

11. The FET biosensor of claim 7,
wherein said melamine antibody comprises a binding region capable of a melamine analog, whereby said melamine molecule and said melamine analog competitively bind to said melamine antibody; and,
whereby binding of said melamine molecule to said melamine antibody produces a first surface charge density on said Silicon channel different and binding of said melamine analog to said melamine antibody produces a second surface charge density, said first and second surface charge density producing different field effects and conductance of said FET biosensor, thereby allowing a user to determine the presence of melamine based on a change of conductance of said FET biosensor.

12. The FET biosensor of claim 11, wherein said melamine analog is BSA-SM2.

13. A direct assay method for measuring the concentration of melamine in a target sample having an unknown amount of melamine, comprising the steps of:
immersing a sensor area of an FET biosensor with a target sample, said FET biosensor comprising, i) a semiconductor substrate; ii) at least one open Silicon channel integral with said semiconductor substrate; iii) a linker molecule; iv) a gate dielectric layer on a surface of said at least one open Silicon channel, said gate dielectric having a surface for attachment of said linker molecule; and, v) a melamine antibody anchored to said gate dielectric via said linker molecule, forming an FET sensor area;
measuring said target signal of said FET biosensor by measuring an electrical signal through said FET biosensor; and,
determining a melamine concentration by comparing said target signal to a standard signal.

14. The method of claim 13 further comprising the steps of:
immersing said sensor area with a standard sample solution of known melamine concentration to produce a standard signal; and,
measuring said standard signal by measuring an electrical signal through said FET biosensor.

15. The method of claim 13 further comprising the steps of:
mixing a known concentration of hapten-melamine to said target sample prior to immersing said sensor area to implement a competitive assay method.

16. The method of claim 13 wherein said target sample comprises dissolved foodstuffs.

17. A competitive assay method for measuring the concentration of melamine in a target sample, the method comprising the steps of:
mixing a reference sample of known concentration of melamine antibody with a target sample having an undetermined concentration of melamine therein producing a testing sample;
immersing a sensor area of an FET biosensor with said testing sample;
measuring a testing sample signal by measuring an electrical signal through said FET biosensor; and,
determining a melamine concentration by comparing said testing sample signal to a standard signal.

18. The method of claim 17 further comprising the steps of:
immersing said sensor area in a standard sample; and,
measuring said standard signal.

19. The method of claim 17 wherein said FET biosensor comprises
a semiconductor substrate;
at least one open Silicon channel integral with said semiconductor substrate;
a linker molecule;
a gate dielectric layer on a surface of said at least one open Silicon channel, said gate dielectric having a surface for attachment of said linker molecule; and,
a melamine antigen anchored to said gate dielectric via said linker molecule forming an FET sensor area for measuring a presence of a target molecule that binds to said melamine antigen.

20. The method of claim 17, wherein said FET biosensor comprises,
a semiconductor substrate;
at least one open Silicon channel integral with said semiconductor substrate;
a linker molecule;
a gate dielectric on a surface of said at least one open Silicon channel, said gate dielectric having a surface for attachment of said linker molecule; and,
a melamine antibody anchored to said gate dielectric via said linker molecule, forming an FET sensor area.

21. The method of claim 17 wherein said melamine antigen is a hapten-melamine molecule.

22. The method of claim 17 wherein said melamine antigen is BSA-SM2.

23. The method of claim 17 wherein said target sample comprises dissolved foodstuffs.
